(19) United States
(12) Patent Application Publication
(10) Pub. No.: US 2023/0406571 A1
(43) Pub. Date: Dec. 21, 2023

(54) FRAME FOR HOLDING SECONDARY PHARMACEUTICAL PACKAGING

(71) Applicant: SCHOTT Pharma Schweiz AG, St. Gallen (CH)

(72) Inventors: Patrick Wolf, St. Gallen (CH); Arne Kloke, St. Gallen (CH); Joel Schnetzer, St. Gallen (CH); Nithan Vasanthamohan, St. Gallen (CH); Belmin Durakovic, St. Gallen (CH)

(73) Assignee: SCHOTT Pharma Schweiz AG, St. Gallen (CH)

(21) Appl. No.: 18/338,447

(22) Filed: Jun. 21, 2023

(30) Foreign Application Priority Data
Jun. 21, 2022 (EP) 22180111.1

(51) Int. Cl. B65D 21/02; B65D 1/36

(52) U.S. Cl. CPC B65D 21/0215; B65D 1/36

(57) ABSTRACT

A frame for holding a secondary pharmaceutical packaging includes: a base plate including at least two openings formed to hold the secondary pharmaceutical packaging; at least two sidewalls connected to the base plate; and at least one stacking element located on the base plate.

### CROSS REFERENCE TO RELATED APPLICATIONS

This application claims priority to European Patent Application No. 22180111.1 filed on Jun. 21, 2022, which is incorporated in its entirety herein by reference.

### BACKGROUND OF THE INVENTION

### 1. Field of the Invention

The invention relates to a frame for holding secondary pharmaceutical packaging, a method for handling frames, a kit of frames, and a use of a frame.

### 2. Description of the Related Art

After manufacturing, pharmaceutical compounds are usually filled and distributed in containers, so-called primary pharmaceutical packaging. This usually involves uncleaned primary pharmaceutical packaging, which needs to be cleaned and sterilized before subsequent filling and sealing. In order to simplify and provide flexibility to this complex process for manufactures of pharmaceutical compounds, pre-cleaned and sterilized primary pharmaceutical packaging are offered in nests or tubs with nests, so-called secondary pharmaceutical packaging. The primary pharmaceutical packaging can be stored in the secondary pharmaceutical packaging. The secondary pharmaceutical packaging can at least in part or fully be enclosed by at least one bag or pouch. These so-called ready-to-use packaging systems may be directly unpacked, filled and resealed by the pharmaceutical company under controlled sterile conditions.

For transport to the pharmaceutical manufacturer, the ready-to-use packaging systems are stacked loosely in a transport box. One problem encountered in the case of these ready-to-use packaging systems is that further automation is hindered. The secondary pharmaceutical packaging has to be removed from the transport box for further processing. The secondary pharmaceutical packaging has to be placed in a different box or holding device for fill and finish, e.g. sealing or packaging, of the primary pharmaceutical packaging.

A further problem that occurs for state of the art ready-to-use packaging systems is that the transport box is disposed after transport to the pharmaceutical manufacturer. The transport box is not suitable for further processing. Thus, for state of the art transport boxes and processes, waste is increasing and reuse over multiple production cycles including the return of secondary pharmaceutical packaging to the manufacturer of primary pharmaceutical packaging is not possible.

A further problem that occurs is that state of the art transport boxes easily deform. In fact, during storage, transport or further processing the transport box changes its shape. This leads to transport damage, e.g. mechanical impact or wear due to protruding, or faults during handling, e.g. jamming.

What is needed in the art is a way to address some of the previously described problems.

### SUMMARY OF THE INVENTION

The previously described problems were extensively investigated and it has been found that, surprisingly, a lack of automation, reuse, and reliability is caused by the handling of secondary pharmaceutical packaging and may be increased by a special frame.

Exemplary embodiments provided according to the present invention thus provide a frame for holding secondary pharmaceutical packaging, a method for handling frames, a kit of frames and a use of a frame which overcome at least some of the previously described problems and which enable further automation of processing primary pharmaceutical packaging, the reuse of secondary pharmaceutical packaging and/or more reliable storage, transport and further processing.

In some embodiments provided according to the present invention, a frame for holding a secondary pharmaceutical packaging includes: a base plate including at least two openings formed to hold the secondary pharmaceutical packaging; at least two sidewalls connected to the base plate; and at least one stacking element located on the base plate.

In some embodiments provided according to the present invention, a method for handling frames is provided. The method includes: i) providing a first frame, the first frame including: a first base plate including at least two openings; at least two first sidewalls connected to the first base plate; and at least one first stacking element located on the first base plate; ii) placing at least one secondary pharmaceutical packaging in the first frame; iii) providing a second frame, the second frame including: a second base plate including at least two openings; at least two second sidewalls connected to the second base plate; and at least one second stacking element located on the second base plate; iv) placing at least one secondary pharmaceutical packaging in the second frame; and v) stacking the second frame on top of the first frame.

In some embodiments provided according to the present invention, a kit of frames includes at least two of the frames described herein, which may be stacked on top of each other.

In some embodiments provided according to the present invention, a frame described herein is used to distribute or store secondary pharmaceutical packaging and optionally primary pharmaceutical packaging.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates a spatial view of an exemplary embodiment of a frame provided according to the invention;

FIG. 2 illustrates a top view of an exemplary embodiment of a frame provided according to the invention; and

FIG. 3 illustrates a side view of an embodiment of a kit of frames provided according to the invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

Exemplary embodiments disclosed herein provide a frame for holding secondary pharmaceutical packaging including a base plate, at least two sidewalls connected to the base plate, and at least one stacking element located on the base plate. The base plate comprises at least two openings and the openings are formed to hold the secondary pharmaceutical packaging.

Exemplary embodiments disclosed herein also provide a method for handling frames comprising the following steps, optionally in this order:
i) providing a first frame described herein,
ii) placing at least one secondary pharmaceutical packaging in the first frame,
iii) providing a second frame described herein,
iv) placing at least one secondary pharmaceutical packaging in the second frame,
v) stacking the second frame on top of the first frame.

Exemplary embodiments disclosed herein also provide a kit of frames comprising at least two frames described herein. Optionally, the frames are stacked on top of each other.

Exemplary embodiments disclosed herein also provide a use of a frame described herein to distribute or store secondary and optionally primary pharmaceutical packaging.

In the present application, all singular terms shall also include the plural and all plural terms shall also include the singular unless otherwise stated. For example, all limitations and exemplary embodiments of a primary/secondary pharmaceutical packaging shall, in particular, also apply to a plurality of, for example, 2 or more primary/secondary pharmaceutical packaging. Furthermore, all limitations and exemplary embodiments of the frame shall also apply to the method, kit and use and vice versa unless otherwise stated.

Frame

The frame provided according to the invention is a frame for holding secondary pharmaceutical packaging comprising a base plate, at least two sidewalls connected to the base plate, at least and one stacking element located on the base plate. The base plate comprises at least two openings and the openings are formed to hold the secondary pharmaceutical packaging.

The frame facilitates further automation, such as filling and finishing, e.g. sealing or packaging, primary pharmaceutical packaging without removal of the secondary pharmaceutical packaging. Further, the frame is robust and flexible and can thus be reused over multiple production cycles or to return primary and/or secondary pharmaceutical packaging for recycling or reuse.

The term “secondary pharmaceutical packaging” herein is to be understood as a nest or a tub comprising a nest, wherein the nest is formed to hold primary pharmaceutical packaging. Optionally, the tub is a tub according to ISO 11040-7:2014(E). Optionally, the nest is a nest selected from the commercially available SCHOTT iQ® platform.

The term “nest” herein refers to an article for holding the primary pharmaceutical packaging. Thus, all primary pharmaceutical packaging are in direct contact with the nest. The nest optionally comprises 10 to 200, optionally 16 to 160, optionally 40 to 100, primary pharmaceutical packaging.

The term “primary pharmaceutical packaging” herein is to be understood as a vial, cartridge, syringe or an ampoule. Primary pharmaceutical packaging is capable of receiving and storing pharmaceutical compounds.

In some embodiments the stacking element is formed to hold a further, optionally identical, frame in place. The stacking elements enable space saving and hold the frames reliable in place due to the restricted movement. Further, this increases the stability of a stack of the frames.

In some embodiments the frame comprises three, optionally four sidewalls. An increased number of sidewalls leads to a higher stability and improved stacking of the frame. Further, mechanical impact on the held secondary pharmaceutical packaging can be reduced. Further, light sensitive pharmaceutical compounds can be processed, as the sidewalls serve as a light shield. Further, contamination can be reduced, as the sidewalls shield against foreign particles. Further, an increased surface area on the side of the frame enables covering a frame with wrapping foil.

In some embodiments the frame comprises 3 to 15, optionally 4 to 14, optionally 5 to 13, optionally 6 to 12, optionally 8 to 10, openings. It may be advantageous to enable the accommodation of state of the art secondary pharmaceutical packaging in the frame. Further, the weight of the frame can be reduced. Further, the placement and removal of the secondary pharmaceutical packaging can be automatized.

In some embodiments the secondary pharmaceutical packaging is a nest or a tub with a nest, optionally wherein the tub is a tub according to ISO 11040-7:2014(E), optionally wherein the nest is formed to hold primary pharmaceutical packaging, optionally wherein primary pharmaceutical packaging is a vial, cartridge, syringe or an ampoule.

In some embodiments the frame comprises 2, optionally 4, optionally 6, optionally 8, optionally 10, stacking elements. An increased number of stacking elements ensures to hold the frame in place during stacking and facilitate improved processing and transport. However, too many stacking elements complicate handling of the frames, e.g. canting, and add to the weight of the frame.

In some embodiments the base plate comprises at least two, optionally four, edges, optionally wherein the two, optionally four, sidewalls are located respectively at an edge, and/or the base plate comprises at least one, optionally four, corner(s).

It may be advantageous to use state of the art pallets, e.g. wood pallets, for transport of the frame. Further, transport damage can be reduced.

The frame may consist of any material. In some embodiments the base plate, the sidewalls and/or the stacking elements consist of the same material. It may be advantageous to use the same material to facilitate reuse and recycling.

In some embodiments the base plate, the sidewalls and/or the stacking elements are connected by a material fit or made from one piece. It may be advantageous to firmly connect the elements of the frame facilitate reuse and recycling.

In some embodiments the base plate comprises an upper surface and a lower surface and/or the base plate comprises notches enclosing each opening, optionally wherein a notch has a width of 8 mm to 16 mm, optionally 10 mm to 12 mm, and/or a depth of 1 mm to 2 mm, optionally 1.4 mm to 1.6 mm. It may be advantageous to enable the accommodation of state of the art secondary pharmaceutical packaging in the frame.

The length, width and height of the frame is not particularly restricted. In some embodiments the frame has a width of 200 mm to 1000 mm, optionally 240 mm to 800 mm, a length of 600 mm to 1400 mm, optionally 800 mm to 1200 mm, and/or a height of 80 mm to 104 mm, optionally 84 mm to 102 mm. It may be advantageous to use state of the art pallets, e.g. wood pallets, for transport of the frame. Further, transport damage can be reduced.

The length and width of an opening is not particularly restricted. In some embodiments, an opening has a width of 160 mm to 240 mm, optionally 190 mm to 210 mm, optionally 202 mm to 208 mm, and/or a length of 190 mm to 270 mm, optionally 220 mm to 250 mm, optionally 235 mm to 240 mm. It may be advantageous to enable the accommodation of state of the art secondary pharmaceutical packaging in the frame.

In some embodiments the sidewalls comprise at least one, optionally 2, optionally 4, holes or cutouts, optionally wherein the holes or cutouts are formed as finger grips. The holes or cutouts can increase the degree of automation, such that machines or workers can reliably hold and process the frames.

In some embodiments the frame comprises, optionally consists of, a polymer, optionally comprises HDPE, PP or ABS, and/or the frame consists of a non-transparent, optionally non-transparent for visible light and/or UV light, material. Durable and robust materials allow for reuse of the frames, without breakage or particle generation. Further, the material is lightweight and recyclable. It may be advantageous to use non-transparent materials for the frame in order to process light sensitive pharmaceutical compounds.

In some embodiments the frame has a rectangular shape and/or the openings have a rectangular shape, optionally with rounded edges. It may be advantageous to use state of the art pallets, e.g. wood pallets, for transport of the frame. Further, transport damage can be reduced.

In some embodiments the stacking element is in form of a notch, optionally embedded in the base plate and optionally located at an edge of the base plate and/or the stacking element is in form of a protrusion, optionally located at an edge of the base plate and/or the stacking element is in form of a thorn, optionally located at a corner of the base plate. Robust stacking elements ensure to hold the frame in place during stacking and facilitate improved processing and transport. Further, visual inspection of the stacking can be simplified, e.g. spaces between stacked frames. Further, the dimensional variance of a stack of frames is decreased.

In some embodiments the base plate comprises an upper surface defining a first surface area and the at least two sidewalls comprise each a lower edge defining a second surface area, wherein the ratio of the first surface area to the second surface area is 0.9 to 1.1, optionally 0.95 to 1.05, optionally 0.97 to 1.03, optionally 0.98 to 1.02, optionally 0.99 to 1.01. An optimized ratio between the first (upper) and second (lower) surface areas facilitates reliable stacking of the frame. An increased amount of room to move leads to wear of the frames and decreases the stability of a stack. However, a very small clearance causes faults during automated processing, e.g. canting. Further, the dimensional variance of a stack of frames is decreased.

In some embodiments the openings are arranged equidistant with respect to the at least one of the two, optionally both, sidewalls or the openings are staggered. It may be advantageous to enable the accommodation of state of the art secondary pharmaceutical packaging in the frame and to increase the accessibility with a defined pattern. Further, the packing density is increased.

In some embodiments the base plate comprises an upper surface, wherein the sidewalls comprise an upper edge and wherein the upper edge is flush with the upper surface. It may be advantageous to avoid unnecessary protrusions, which could cause wear on the frames, e.g. generate particles, or lead to faults during automated processing due to canting. Further, the stability of a stack of frames is increased.

Method

The method provided according to the invention is a method for handling frames comprising the following steps, optionally in this order:
i) providing a first frame described herein,
ii) placing at least one secondary pharmaceutical packaging in the first frame,
iii) providing a second frame described herein,
iv) placing at least one secondary pharmaceutical packaging in the second frame,
v) stacking the second frame on top of the first frame.

In some embodiments the method further comprises the step(s): placing at least one primary pharmaceutical packaging in a secondary pharmaceutical packaging, and optionally filling the primary pharmaceutical packaging, optionally with a pharmaceutical substance and/or optionally inspecting the primary pharmaceutical packaging.

In some embodiments the method further comprises the step(s): sealing and/or enclosing the secondary pharmaceutical packaging, optionally with a pouch and/or at least one, optionally two, bag(s), optionally before placing the secondary pharmaceutical packaging in the first and/or second frame, and optionally removing a pouch and/or at least one, optionally two, bag(s) from the secondary pharmaceutical packaging.

In some embodiments the method further comprises the step: removing the secondary pharmaceutical packaging from the first and/or second frame.

In some embodiments the method further comprises the step: placing a cover plate on top of the second frame.

In some embodiments the method further comprises the step stacking further frames described herein on top of the second frame and/or below the first frame.

Kit

The kit provided according to the invention is a kit of frames comprising at least two frames described herein, optionally the frames are stacked on top of each other.

The kit facilitates further automation, such as filling and finishing, e.g. sealing or packaging, primary pharmaceutical packaging without removal of the secondary pharmaceutical packaging. Further, the kit enables the reuse of frames over multiple production cycles or the return of primary and/or secondary pharmaceutical packaging for recycling or reuse.

In some embodiments the kit further comprises at least one secondary pharmaceutical packaging, optionally held by a frame, and optionally further comprising primary pharmaceutical packaging, optionally stored in the secondary pharmaceutical packaging, and optionally wherein the secondary pharmaceutical packaging is sealed by a pouch and/or enclosed by one, optionally two, bag(s).

In some embodiments the kit comprises 4 to 80, optionally 10 to 50, optionally 20 to optionally 25 to 30, frames. It may be advantageous to use state of the art pallets, e.g. wood pallets, for transport of the frame. Further, transport damage can be reduced. Further, it may be advantageous to enable the accommodation of state of the art secondary pharmaceutical packaging in the frame.

The length, width and height of the kit is not particularly restricted. In some embodiments the kit has a width of 600 mm to 1000 mm, optionally about 800 mm, a length of 1000 mm to 1400 mm, optionally about 1200 mm, and/or a height of 1400 mm to 1800 mm, optionally about 1600 mm. It may be advantageous to use state of the art pallets, e.g. wood pallets, for transport of the frame. Further, transport damage can be reduced. Further, it may be advantageous to enable the accommodation of state of the art secondary pharmaceutical packaging in the frame.

In some embodiments the kit further comprises at least one cover plate, optionally wherein the cover plate is formed to be placed on the base plate, optionally further comprising wrapping foil, optionally enclosing the frames and the cover plate. The cover plate can further protect the primary and/or secondary pharmaceutical packaging. Additionally, the cover plate can enable processing of light sensitive pharmaceutical compounds.

In some embodiments the frames are arranged in 1 to 5 stacks, optionally 2 to 4 stacks, optionally 3 and optionally wherein one stack consists of 8 to 20, optionally 10 to 16, optionally 12 to 14, frames. It may be advantageous to use state of the art pallets, e.g. wood pallets, for transport of the frame. Further, transport damage can be reduced. Further, it may be advantageous to enable the accommodation of state of the art secondary pharmaceutical packaging in the frame.

In some embodiments the kit comprises at least one pouch, wherein the pouch covers an opening, optionally wherein the pouch is connected to the base plate. It may be advantageous to seal the secondary packing with the aid of the base plate. This eases further processing, such as unpacking pre-sterilized primary and/or secondary pharmaceutical packaging. Further, sealing on an increased area of a flat surface is more reliable and further bags or pouches can be omitted.

Use

The use provided according to the invention is a use of a frame described herein to distribute or store secondary and optionally primary pharmaceutical packaging.

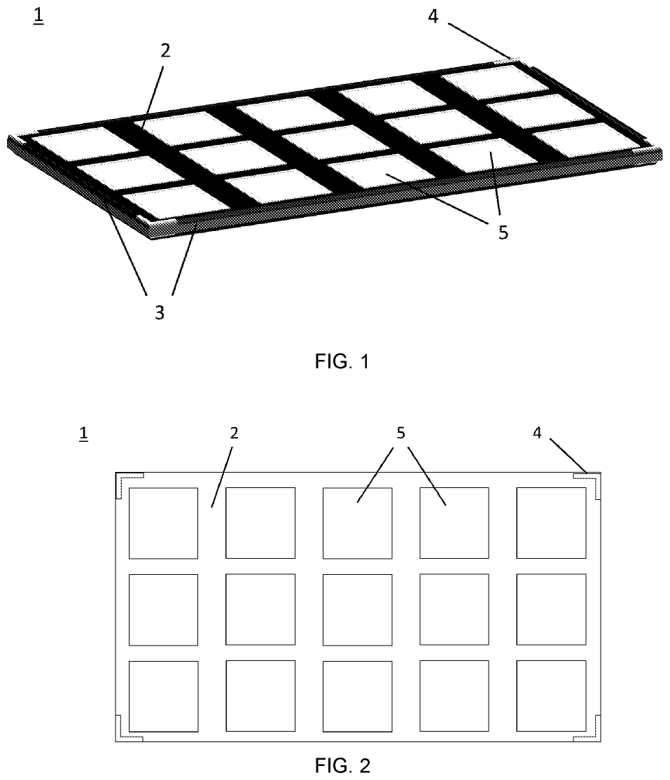

Referring now to the drawings, FIG. 1 shows a spatial view of an exemplary embodiment of a frame 1 provided according to the invention and FIG. 2 shows a top view of an exemplary embodiment of a frame 1 provided according to the invention. As can be seen from FIGS. 1 and 2, the frame 1 comprises a base plate 2, at least two sidewalls 3 (not numbered in FIG. 2) connected to the base plate 2, and at least one stacking element 4 located on the base plate 2. The base plate 2 comprises at least two openings 5 and the openings 5 are formed to hold the secondary pharmaceutical packaging 6.

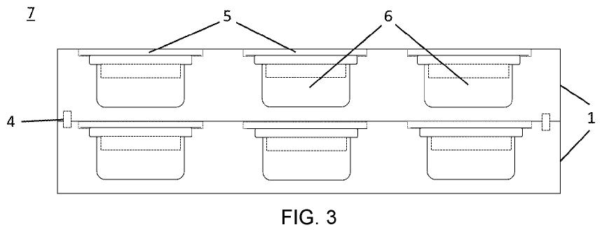

FIG. 3 shows a cross-sectional side view of an exemplary embodiment of a kit of frames 7 provided according to the invention. As can be seen from FIG. 3, the kit comprises at least two frames 1 described herein, for example as depicted in FIGS. 1 and 2, wherein the frames 1 are stacked on top of each other. The kit comprises secondary pharmaceutical packaging 7, for example a tub comprising a nest. The stacking elements 4 hold the respective frame in place.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE NUMERALS

1 Frame
2 Base plate
3 Sidewalls
4 Stacking element
5 Openings
6 Secondary pharmaceutical packaging
7 Kit of frames

What is claimed is:

1. A frame for holding a secondary pharmaceutical packaging, the frame comprising:
a base plate comprising at least two openings formed to hold the secondary pharmaceutical packaging;
at least two sidewalls connected to the base plate; and
at least one stacking element located on the base plate.

2. The frame of claim 1, wherein the at least one stacking element is formed to hold a further frame in place.

3. The frame of claim 1, wherein the at least two sidewalls comprises three or four sidewalls.

4. The frame of claim 1, wherein the at least two openings comprise 3 to 15 openings.

5. The frame of claim 1, wherein the secondary pharmaceutical packaging is a nest or a tub with a nest.

6. The frame of claim 1, wherein the at least one stacking element comprises a plurality of stacking elements.

7. The frame of claim 1, wherein the base plate comprises at least two edges and/or at least one corner.

8. The frame of claim 1, wherein the base plate, the at least two sidewalls and/or the at least one stacking element consist of the same material.

9. The frame of claim 1, wherein the base plate, the at least two sidewalls and/or the at least one stacking element are connected by a material fit or made from one piece.

10. The frame of claim 1, wherein the base plate comprises at least one of:
an upper surface and a lower surface; or
notches enclosing each of the at least two openings.

11. The frame of claim 1, wherein the frame has a width of 200 mm to 1000 mm, a length of 600 mm to 1400 mm, and/or a height of 80 mm to 104 mm.

12. The frame of claim 1, wherein at least one of the at least two openings has a width of 160 mm to 240 mm and/or a length of 190 mm to 270 mm.

13. The frame of claim 1, wherein the at least two sidewalls comprise at least one hole or cutout.

14. The frame of claim 1, wherein the frame comprises a polymer and/or the frame consists of a non-transparent material.

15. The frame of claim 1, wherein the frame has a rectangular shape and/or the at least two openings have a rectangular shape.

16. The frame of claim 1, wherein the at least one stacking element is in the form of at least one of the following:
a notch;
a protrusion; or
a thorn.

17. The frame of claim 1, wherein the base plate comprises an upper surface defining a first surface area and the at least two sidewalls each comprise a lower edge defining a second surface area, wherein a ratio of the first surface area to the second surface area is 0.9 to 1.1.

18. The frame of claim 1, wherein the at least two openings are arranged equidistant with respect to at least one of the at least two sidewalls or the at least two openings are staggered.

19. The frame of claim 1, wherein the base plate comprises an upper surface, the at least two sidewalls comprise an upper edge, and the upper edge is flush with the upper surface.

20. A method for handling frames, the method comprising:
i) providing a first frame, the first frame comprising:
a first base plate comprising at least two openings;
at least two first sidewalls connected to the first base plate; and
at least one first stacking element located on the first base plate;
ii) placing at least one secondary pharmaceutical packaging in the first frame;
iii) providing a second frame, the second frame comprising:
a second base plate comprising at least two openings;
at least two second sidewalls connected to the second base plate; and
at least one second stacking element located on the second base plate;
iv) placing at least one secondary pharmaceutical packaging in the second frame; and
v) stacking the second frame on top of the first frame.
